(19) United States
(12) Patent Application Publication
(10) Pub. No.: US 2015/0072357 A1
(43) Pub. Date: Mar. 12, 2015

(54) Cholesteryl Succinyl Silane Bound Proteins and Methods For Producing and Using the Same

(71) Applicants: Xiaoyi Wu, Tucson, AZ (US); Celine Cohn, Tucson, AZ (US); Ryan Norrix, Tucson, AZ (US)

(72) Inventors: Xiaoyi Wu, Tucson, AZ (US); Celine Cohn, Tucson, AZ (US); Ryan Norrix, Tucson, AZ (US)

(73) Assignee: THE ARIZONA BOARD OF REGENTS ON BEHALF OF THE UNIVERSITY OF ARIZONA, Tucson, AZ (US)

(21) Appl. No.: 14/478,235

(22) Filed: Sep. 5, 2014

Related U.S. Application Data
(60) Provisional application No. 61/874,419, filed on Sep. 6, 2013.

(51) Int. Cl. G01N 33/543; G01N 33/574

(52) U.S. Cl. CPC G01N 33/54353; G01N 33/57492; G01N 33/57415; G01N 33/57426
USPC 435/7.23; 264/465

(57) ABSTRACT

The present invention provides a various three-dimensional polymeric scaffold and methods for producing and using the same. In one embodiment, the three-dimensional polymeric scaffold is used to promote cell migration. Yet in another embodiment, the three-dimensional polymeric scaffold comprises enhanced surface functionalization. In one particular embodiment, the polymeric scaffold comprises a plurality of polymer layers, wherein each polymer layer comprises microchannels. Still in another particular embodiment, the composition comprises a fibrous cholesteryl succinyl silane. Yet in another particular embodiment, a polymer comprising cholesteryl succinyl silane attached (e.g., hybridized) to its surface is provided.

### CROSS-REFERENCE TO RELATED APPLICATIONS

This application claims the priority benefit of U.S. Provisional Application No. 61/874,419, filed Sep. 6, 2013, which is incorporated herein by reference in its entirety.

### STATEMENT REGARDING FEDERALLY FUNDED RESEARCH

This invention was made with government support under Grant Nos. T32 HL007955 and R21 EB009160, awarded by the National Institutes of Health (NIH). The government has certain rights in the invention.

### FIELD OF THE INVENTION

The present invention relates to a non-woven polymer fiber that comprises cholesteryl succinyl silanes and method for producing and using the same. In one particular embodiment, the non-woven-polymer fiber of the invention comprises a nanostructured cholesteryl succinyl silanes that are configured to immobilize proteins, such as peptides, antibodies etc.

### BACKGROUND OF THE INVENTION

Various proteins and peptides, such as antibodies, are increasingly used as the detection element in increasingly sensitive biosensors and drug delivery systems. These systems often have small surface areas which demand functionalization to occur with extreme efficiency. However, protein (in particular antibody) immobilization is still often achieved by hydrophobic interactions, which lead to protein denaturation and leave fewer than 5% of the binding sites available for antigen capture. Such a low amount of proteins for binding renders these systems relatively ineffective in detecting antigens.

Accordingly, there is a need for a system that can provide a selective and/or efficient antigen capture.

### SUMMARY OF THE INVENTION

On cell membranes, cholesterol plays a large part in immobilizing membrane-bound proteins. The small head groups and sterol rings found within the cholesterol molecule promotes the formation of liquid ordered micro-domains. Thus, some aspects of the invention provide a system or a composition that comprises cholesteryl succinyl silane (CSS) that can effectively mimic naturally-occurring cholesterol micro-domains.

In particular, the present inventors have discovered that CSS is capable of promoting strong antibody (“AB”) orientation and capture a significantly more (e.g., 3× or more, typically, 5× or more, and often 6× or more, and most often 7× more) cells than antibodies immobilized via hydrophobic interactions. In some embodiments of the invention, cholesteryl succinyl silane is attached to a non-woven polymer fiber (e.g., to a polycaprolcatone (PCL) backbone) to provide immobilization of proteins including antibodies. Without being bound by any theory, it is believed that attachment of CSS (e.g., via hybridization) to a non-woven polymer fiber allows for the strong functionalization or immobilization of proteins promoted by CSS while maintaining strong mechanical properties that is provided by the non-woven polymer fiber, such as PCL backbone.

One particular aspect of the invention provides a composition comprising: a nanostructure cholesteryl succinyl silane (CSS); and an antibody immobilized on said nanostructure CSS. In some embodiments, said antibody is adapted for selectively capturing a cell. Within these embodiments, in some instances, the cell is a circulating tumor cell. Exemplary circulating tumor cells that can be captured by the compositions and methods of the invention include, but are not limited to, breast cancer cell, leukemic cell, lymphoma cancer cell, metastatic breast cancer (MBC) cells, as well as any other metastatic and non-metastatic cancer cells that can be captured from a fluid sample of the subject. Exemplary fluid samples of the subject that are useful include, but are not limited to, blood, serum, spinal fluid, urine, sputum, mucus, saliva and any other bodily fluid that can contain cells. In one particular embodiment, the circulating tumor cells include MBC cells, metastatic pancreatic cancer cells; non-metastatic pancreatic cancer cells; metastatic prostate cancer cells; non-metastatic prostate cancer cells; metastatic lung cancer cells; non-metastatic lung cancer cells; non-metastatic bladder cancer cells; colorectal cancer cells; gastric cancer cells, etc.

In other embodiments, the composition further comprises a non-woven polymer fiber, wherein said nanostructure CSS is attached to said non-woven polymer fiber. In some instances, the non-woven polymer fiber is a plasma treated non-woven polymer fiber. In one particular instance, the non-woven polymer fiber is air-plasma treated.

Still in other embodiments, the diameter of said non-woven polymer fiber is substantially similar to extracellular matrix. Yet in other embodiments, said non-woven polymer fiber is made from a polymer comprising poly(caprolactone) (“PCL”), polyvinyl alcohol (“PVA”), polylactic acid (“PLA”), a copolymer comprising the same, or any combination thereof.

Yet still in other embodiments, a monomeric unit of said cholesteryl succinyl silane is of the formula: A-B-C, wherein A is cholesterol, B is succinyl unit, and C is a silane unit. In some instances, said silane unit is a moiety of the formula: —R^{a}—Si(OR^{b})₃, wherein R^{a} is alkylene, and each of R^{b} is independently hydrogen or alkyl.

Another aspect of the invention provides a method for producing a composition comprising: a non-woven polymer fiber; a nanostructure cholesteryl succinyl silane (CSS) attached to said non-woven polymer fiber; and an antibody immobilized on said CSS. Such a method typically includes (i) producing a non-woven polymer fiber; (ii) contacting said non-woven polymer fiber with a solution of monomeric cholesteryl succinyl silane under conditions sufficient to produce a nanostructure cholesteryl succinyl silane attached to said non-woven polymer fiber; and (iii) immobilizing an antibody to said nanostructure cholesteryl succinyl silane to produce said composition. In one embodiment, said non-woven polymer fiber is produced by electrospinning process. Still in other embodiments, the diameter of said non-woven polymer fiber is substantially similar to extracellular matrix. Yet in other embodiments, said non-woven polymer fiber comprises PCL, PVA, PLA, a copolymer comprising the same, or any combination thereof. In some instances, the polymer is treated with plasma before being contacted with a monomeric CSS solution. Generally, the monomeric CSS solution is an acidic aqueous solution.

Yet another aspect of the invention provides a method for producing a composition comprising a nanostructure cholesteryl succinyl silane (CSS) and an antibody immobilized on said nanostructure CSS. Such a method typically comprises: providing an acidic solution of cholesteryl succinyl silane having a critical micelle concentration; producing a nanostructure CSS from said acidic solution of cholesteryl succinyl silane using an electrospinning process; and immobilizing an antibody to said nanostructure cholesteryl succinyl silane to produce said composition. The term “critical micelle concentration” or “CMC” refers to a concentration of CSS at or above which micelle of CSS forms. Typically CMC depends on the particular CSS compound.

Still another aspect of the invention provides a method for detecting the presence of a cancer in a subject. In one particular embodiment, said method comprises (i) contacting a fluid sample of the subject with a composition comprising: a nanostructure cholesteryl succinyl silane (CSS); and a cancer cell antibody immobilized on said CSS; and determining the formation of a complex between said cancer cell antibody and a cancer cell, wherein the presence of said complex is an indication that said subject has a cancer. In some embodiments, said cancer cell comprises a circulating cancer cell. In other embodiments, said composition further comprises a non-woven polymer fiber, wherein said nanostructure CSS is attached to said non-woven polymer fiber.

Another aspect of the invention provides a polymer comprising a nanostructure cholesteryl succinyl silane (CSS). In some embodiments, the polymer further comprises an antibody immobilized to CSS.

It should be appreciated that while the present invention is described in reference to using an antibody as the immobilized protein, in general the scope of the invention encompasses any protein that can selective bind to a particular ligand.

Still another aspect of the invention provides a composition comprising a plurality of fibrous polymer layers, wherein each layer of said fibrous polymer comprises microchannels. In some embodiments, fibers of said fibrous polymer layer are aligned. In other embodiments, fibers of said fibrous polymer layer are randomly oriented. Yet in other embodiments, the top layer of said fibrous polymer layer further comprises cells. Such a composition can be used to promote cellular migration and the transportation of nutrient/waste through the scaffold. Multiple layers of electrospun scaffolds (or layers) are typically welded or bound together (e.g., using an adhesive or by treating each layer with plasma to allow direct adhesion between two or more layers), creating a singular construct with multiple cell seeding layers/microenvironments. This construct design allows for quick and cheap fabrication of 3D electrospun constructs.

### BRIEF DESCRIPTION OF THE DRAWINGS

FIG. 1A is a schematic illustration depicting promotion of proper antibody orientation by CSS micro-domains.

FIG. 1B is a bar graph showing experimental results of cell capture by antibody is significantly increased in the presence of CSS.

FIG. 1C is a bar graph showing that hybridized fibers also immobilize proteins via micro-domain interactions.

FIG. 2 a graph showing comparative results of the three electrospun materials (PCL, plasma-treated PCL, and CSS) that were exposed to BSA. Protein aggregation was seen within the PCL samples. Even though no BSA was added over the course of the study, the fluorescent intensity increased indicating protein aggregation.

FIG. 3 is a bar graph showing the results of cell capture experiments for the three electrospun scaffolds. As can be seen, electrospun CSS fibers were able to capture significantly more cells than either PCL or Plasma-treated PCL fibers (p<0.005).

FIG. 4 is schematic illustration of antibody immobilization on PCL (top left); Plasma-treated PCL (top right); CSS (bottom left); and CSS cross-sectional close-up illustration (bottom right).

FIG. 5 is a graph showing the result of DiO immobilization on CSS fibers.

FIG. 6 is a graph of the result of DiO immobilization on PCL air-plasma treated fibers, PCL incubated with CSS polymer, and PCL incubated with CSS monomer.

FIG. 7 is a graph showing the result of Ganta-22 cell capture experiment on various polymers and polymers comprising CSS nanostructure.

FIG. 8 is a graph showing the results of MDA-MB-231 cells capturing experiment using CSS chips with immobilized antibodies.

### DETAILED DESCRIPTION OF THE INVENTION

### Definitions

“Alkyl” refers to a saturated linear monovalent hydrocarbon moiety of one to twenty, typically one to twelve, and often one to six, carbon atoms or a saturated branched monovalent hydrocarbon moiety of three to twenty, typically three to twelve, and often three to six, carbon atoms. Exemplary alkyl group include, but are not limited to, methyl, ethyl, n-propyl, 2-propyl, tert-butyl, pentyl, and the like.

“Alkylene” refers to a saturated linear divalent hydrocarbon moiety of one to twenty, typically one to twelve, and often one to six, carbon atoms or a branched saturated divalent hydrocarbon moiety of three to twenty, typically three to twelve, and often three to six, carbon atoms. Exemplary alkylene groups include, but are not limited to, methylene, ethylene, propylene, butylene, pentylene, and the like.

“ECM” refers to extracellular matrix. Extracellular matrix (ECM) is a collection of extracellular molecules secreted by cells that provides structural and biochemical support to the surrounding cells.

“Immobilized” means the amount of protein (or any other ligand) that is attached to CSS does not change significantly under the given conditions (e.g., assay or diagnostic conditions). It should be appreciated the protein may be bound to CSS or an equilibrium is reached between protein and CSS such that a steady-state concentration (or amount) of protein remains associated with CSS. Typically, the term “immobilized” means that the amount of protein that is associated with and/or bound to CSS remains substantially the same under the given condition. For example, in one particular embodiment, the term “immobilized” means that the amount or the level of protein that is associated with and/or bound to CSS remains constant within at least 80%, typically at least 90%, often at least 95%, and more often at least 98% under the assay or diagnostic conditions that are used.

Unless the context requires otherwise, the term “attached” refers to covalent attachment, ionic attachment, attachment due to the presence of Van Der Waal's interaction, and/or any other means of having the receptor being present within the vicinity of the cholesteryl succinyl silane that is present in the composition of the invention. Typically, the term “attached” means that the protein or any other receptor that is attached to and/or associated with the cholesteryl succinyl silane moiety.

When referring to a diameter of the polymer, in particular non-woven polymer fiber, the term “substantially similar to extracellular matrix” means the diameter of the polymer is no more than ±20%, typically no more than ±10%, and often no more than ±5% compared to the average size of the ECM.

Unless the context requires otherwise, the term “selective”, when referring to a ligand-receptor complex formation means that the protein has at least 80% selectivity, typically at least 90% selectivity, often at least 95% selectivity, and most often at least 98% selectivity for a particular ligand.

Unless the context requires otherwise, the term “ligand” refers to any substance that is capable of binding selectively with a receptor. A ligand can be an antigen, an antibody, an oligonucleotide, an oligopeptide (including proteins, hormone, etc.), an enzyme, a substrate, a drug, a drug-receptor, cell surface, receptor agonists, partial agonists, mixed agonists, antagonists, response-inducing or stimulus molecules, drugs, hormones, pheromones, transmitters, autacoids, growth factors, cytokines, prosthetic groups, coenzymes, cofactors, substrates, precursors, vitamins, toxins, regulatory factors, antigens, haptens, carbohydrates, molecular mimics, structural molecules, effector molecules, selectable molecules, biotin, digoxigenin, cross-reactants, analogs, competitors or derivatives of these molecules as well as library-selected non-oligonucleotide molecules capable of selectively binding to selected targets and conjugates formed by attaching any of these molecules to a second molecule, and any other molecule that binds selectively with a corresponding receptor.

Unless the context requires otherwise, the term “receptor” refers to any substance that can selectively bind with or bind to a corresponding ligand. A receptor can be an antigen, an antibody, an oligonucleotide, an oligopeptide (including proteins, hormone, etc.), an enzyme, a substrate, a drug, a drug-receptor, cell surface, and any other molecule that binds selectively with a corresponding ligand.

It should be appreciated that the terms “ligand” and “receptor” do not refer to any particular substance or size relationship. These terms are only operational terms that indicate selective binding between the ligand and the corresponding receptor. Any protein that is immobilized on CSS can be referred to as either the receptor or the ligand. In general, however, for the sake of brevity and consistency the moiety or the protein that is attached to or immobilized onto CSS is typically referred to herein as a “receptor”. Thus, if an antibody is attached to or immobilized onto CSS, the antibody is a receptor and the corresponding antigen or the cell that the antibody selectively binds to is referred to as a ligand. However, if an antigen or a cell is attached to or immobilized onto CSS, then the antigen is a receptor and the corresponding antibody is a ligand. Thus, the terms “ligand” and “receptor”, for the purpose of this invention, is a purely operational terms and do not constitute any limitation of the moiety used.

### Compositions of the Invention

The composition of the invention can be used to immobilize or attach any protein of interest. Such a protein is typically immobilized onto a cholesteryl succinyl silane that is attached to a non-woven polymer fiber. However, for the sake of brevity and clarity, the present invention will now be described with reference to immobilizing an antibody. It should be appreciated that the scope of the invention is not limited to immobilizing or attaching an antibody to a non-woven polymer fiber comprising a cholesteryl succinyl silane.

Antibodies are increasingly used as the detection element in increasingly sensitive biosensors and drug delivery systems. These systems often have small surface areas which demand functionalization to occur with extreme efficiency. Without being bound by any theory, it is believed that conventional methods for immobilizing an antibody is typically achieved by hydrophobic interactions, which is believed to result in protein denaturation and often leaving fewer than 5% of the binding sites available for antigen capture.

Some aspects of the invention are based on the discovery by the present inventors that use of cholesterol significantly reduces or eliminates the problems associated with antibody immobilization observed in conventional methods. On cell membranes, cholesterol plays a large part in immobilizing membrane-bound proteins. The small head groups and sterol rings found within the cholesterol molecule promotes the formation of liquid ordered micro-domains. The present inventors have discovered that cholesteryl succinyl silane (CSS) can be electrospun into fibers that mimic naturally-occurring cholesterol micro-domains. The present inventors have shown that CSS is capable of promoting strong antibody (“AB”) orientation and capture at least 5 times, typically at least 6 times, and often at least 7 times more cells than antibodies immobilized via hydrophobic interactions. Accordingly, one particular embodiment of the invention provides cholesteryl succinyl silane (CSS) that is attached to a polycaprolcatone (PCL) or other similar polymeric backbone. This hybridization (or attachment) allows for the strong functionalization promoted by CSS while maintaining strong mechanical properties, provided by the PCL backbone. Thus, the present inventors have found that in one particular embodiment, the hybridized PCL:CSS electrospun fibers are strongly suited for efficient antibody immobilization. Without being bound by any theory, it is believed that cholesterol succinyl silane (CSS) promotes proper antibody orientation while PCL provides mechanical integrity. This hybridized material can be used in diagnostic, drug delivery, and biosensor systems in which antibody orientation is an important component.

Other suitable substitute groups for the hydrophilic head of CSS include, but are not limited to, 3-Aminopropyltriethoxysilane; 3-Aminopropyltrimethoxysilane; 3-aminopropyl methyldiethoxy silane; N-(amino-ethyl)-amino-propyl trimethoxy silane; N-(2-Aminoethyl)-3-Aminopropyltriethoxysilane; 3-(2-Aminoethylamino)propyl-dimethoxymethylsilane; 3-(2-Aminoethylaminopropylmethyldimethoxysilane; Anilino-methyl-triethoxysilane; N-(3-Triethoxysilylpropyl)ethylenediamine; 3-Isocyanatopropyltriethoxysilane; as well as other similar compounds known to one skilled in the art. Suitable substitute groups for the hydrophobic tail of CSS include, but are not limited to, carboxyl cholesterol; cholesteryl hemisuccinate; amino cholesterol; aldehyde cholesterol; thiocholesterol; isocyanate cholesterol; and other similar cholesterol derivatives known to one skilled in the art.

A typical process for producing a composition comprising a non-woven polymer fiber, a cholesteryl succinyl silane attached to the polymer fiber is illustrated with reference to attaching the CSS to a poly(caprolactone) (“PCL”) fiber. Thus, a 10% w/v PCL solution was prepared in hexafluoro-propanol (HFP) and then electrospun. The PCL fibers then underwent an air-plasma treatment for ten minutes and soaked overnight in a various solution concentrations (e.g., 0.1%, 1.0%, and 5.0% w/v) of CSS in ethanol and water. Energy-dispersive spectroscopy (EDS) was used to confirm the formation of the hybridized fibers, i.e., polymer fibers with CSS attached. The immobilization capabilities of the hybridized fibers were compared to electrospun PCL fibers. Dioctadecyloxacarbocyanine perchlorate (DiO) was used as a model system to understand the immobilization efficiency of the electrospun fibers, as DiO has two long carbon chains that act as transmembrane components. Furthermore, half of the fibers underwent a bovine serum albumin pretreatment, which would block hydrophobic regions of the fibers. This was used to confirm that DiO was directly interacting with the fibers. DiO immobilization was repeated after hybrid fiber shelf storage for 5 months to determine if the material was “shelf stable”.

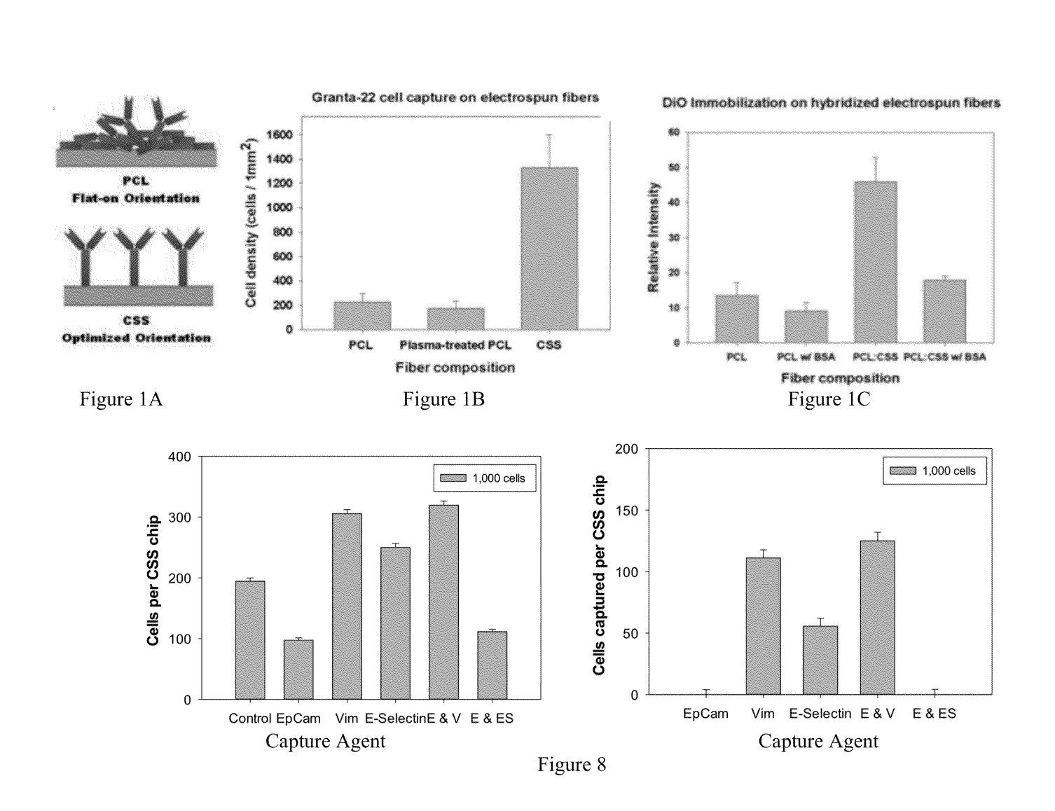

When PCL and CSS have similar amounts of antibody immobilized, CSS was still able to capture 5× more cells than PCL (FIG. 1B). This result indicates that CSS promotes proper AB orientation (FIG. 1A). In order to improve the mechanical properties of CSS, the cholesterol-based material was conjugated or attached to a PCL backbone. EDS was used to confirm the formation of a hybridized fiber. Without begin bound by any theory, it is believed that DiO immobilization on the hybridized PCL:CSS fibers occurs due to interactions with micro-domains (FIG. 1C). If only hydrophobic interactions were responsible for DiO immobilization, similar levels of immobilization would have been seen for the PCL and hybridized fibers. Hybridized fibers that were stored for 5 months were able to immobilize comparable levels of DiO as freshly synthesized fibers.

As the results indicate, hybridized PCL:CSS electrospun fibers are strongly suited for highly efficient antibody immobilization (FIG. 1B). It is believed that CSS promotes proper antibody orientation via micro-domains while PCL provides mechanical integrity. This hybridized material can be used in a wide variety of applications including, but not limited to, drug delivery and biosensor systems which require a substantially proper antibody orientation. In addition, the lipid nanofibers allowed immobilization of cocktails of cell-capture: agents. Numerous antibodies or capture proteins can be integrated on the same surface, allowing for the simultaneous detection of multiple antigens.

Antibodies that can be immobilized by the composition of the invention can be produced by a variety of methods known to one skilled in the art. For example, typically in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature, 1975, 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

In addition, some of the suitable antibodies, such as those of various cancer cells, are already available. Thus, various antibodies that can be immobilized in the composition of the invention can be readily obtained by the procedures known to one skilled in the art or are already available.

### Three-Dimensional Polymeric Scaffold Comprising Microchannels

Numerous disease states exist in which tissue is damaged and needs replacement. Injured tissue can often exacerbate tissue damage, as the remaining, healthy tissue is faced with an increased workload. Regeneration of tissue needs a compliant three-dimensional (3D) scaffold that does not elicit an immune response from the host. Furthermore, the construct needs to encourage nutrient perfusion, in turn promoting cellular proliferation throughout the artificial scaffold. Such a 3D construct could be used to replace numerous types of damaged tissues. While some aspects of the invention are useful in a wide variety of tissue regeneration and in vitro cell studies, for the sake of brevity and clarity this aspect of the invention will now be described for cardiac tissue regeneration.

Myocardial infarction leads to more than 600,000 deaths per year in the United States. Even if a patient survives a heart attack, these events often leave large portions of the heart muscle incapable of generating force, ultimately leading to heart failure. Research is now underway to engineer scaffolds that can be implanted in heart muscle to promote regeneration of functional myocardial tissue. Engineering constructs from electrospun fibers is promising because the resulting morphology strongly mimics the extracellular matrix. However, electrospun scaffolds form a thin, two-dimensional sheet that is unsuited for the repair of 3D defects.

Accordingly, some aspects of the invention provide three-dimensional (3D) construct from stacked electrospun scaffolds that include microchannels to increase nutrient perfusion and cellular proliferation through stacked electrospun scaffolds. The present inventors have discovered that the inclusion of microchannels results in higher cell densities throughout the 3D construct. Furthermore, the stacked electrospun scaffolds were welded together at distinct points, allowing for a 3D construct from 2D components. Welding the electrospun scaffolds together also helps ensure that the individual layers do not delaminate from one another, possibly reducing an inflammatory response when used in vivo. Such a system can be used for three-dimensional tissue regeneration.

Three-dimensional cardiac constructs have strong market potential as many post-myocardial infarcted patients are left with compromised cardiac tissue. The loss of perfusion due to myocardial infarction (MI) leads to a percentage of necrotic cardiac tissue. The necrotic portion of the myocardium strongly affects the ventricular output of the heart and can often lead to heart failure. Numerous 2D cardiac patches have been designed that attempt to strengthen and/or recellularize the affected tissue. However, these solutions leave the necrotic tissue intact, ultimately hindering the ability of the heart to properly perform.

Some aspects of the invention provide a three-dimensional polymeric construct that can be used to replace the necrotic tissue and help re-establish proper ventricular output/cardiac function. In some embodiments, a three-dimensional construct was engineered that can be used to replace damaged tissue and promote tissue regeneration. The construct has a number of variables that can be customized depending on the type of tissue that is being replaced. In one particular embodiment, the polymeric construct is useful for cardiac tissue regeneration. In one particular example, aligned electrospun fibers were produced and cut into scaffolds with a diameter of 8 mm. The individual scaffold layers were then perforated with microchannels (to allow for nutrient perfusion through the 3D construct). Multiple electrospun layers were then stacked one on top the other to create a 3D construct. The scaffold layers were then welded together at discrete points along the outer perimeter of the construct. Cells have been seeded on the top layer of the construct (cell proliferation throughout the construct is encouraged by the inclusion of the microchannels and the use of a custom-built bioreactor). Such a scaffold offers surgeons a way to replace necrotic tissue with a cellularized scaffold that can promote tissue regeneration.

Conventional patches that are commercially available are considered to be 2D patches that can be used to encourage cellular proliferation into damaged tissue. However, many of these patches have limited physiological benefit because the damaged tissue is still present (causing increased work load for any remaining healthy muscle). The 3D construct of the present invention allows surgeons to actively remove the damaged muscle and provide a robust scaffold for tissue regeneration. Even though construct utilization is invasive (surgical removal of necrotic tissue), the construct of the invention leads to increased ventricular output, repairing much of the damage caused by loss of perfusion that occurs during MI.

In some embodiments, a number of engineering principles have been incorporated to design a construct that can be used to completely replace necrotic tissue. Exemplary engineering principles utilized include one or more of the following:
Electrospun polymer fibers: These nano- and micro-scale fibers strongly mimic the extracellular matrix.
Aligned or randomly oriented fibers: The ability to control the orientation of the fiber allows methods of the invention to create 3D scaffolds whose ECM closely resembles that seen in nature (e.g., aligned for cardiac regeneration, random for some smooth muscles). Orientation of fibers helps cellular organization and function. For example, aligned fibers for cardiac regeneration allows scaffold compliance to allow the seeded myocytes to beat properly.
Microchannels: The inclusion of microchannels helps promote cellular migration/proliferation throughout all the layers of the 3D construct. Microchannels also help with nutrient perfusion through the construct.
Welding stacked layers: Even though electrospinning produces scaffolds that strongly mimic the natural ECM, a well-documented downfall is that the resulting scaffolds lack the thickness needed for many clinical applications. Welding the individual electrospun layers together also minimizes the abrasiveness of the construct boundaries, thereby significantly reducing or minimizing the risk of an inflammatory response.

Many existing technologies are based on 2D patches that help strengthen the tissue and/or increase the number of viable cells within the tissue. However, these systems are flawed in that the necrotic tissue is still present, allowing for continued weakening of the surrounding tissue (due to increased demand placed on the healthy tissue). The construct of the invention includes a number of engineering principles to build a 3D scaffold that can be used to replace damaged tissue throughout the body. First, polymer nano- and micro-scale fibers are electrospun, producing 2D scaffolds that strongly mimic the extracellular matrix. Second, either aligned or randomly oriented fibers are produced as desired. This control of fiber orientation allows creation of 3D scaffolds whose ECM closely resembles that seen in nature. The inclusion of microchannels helps promote cellular migration/proliferation and nutrient perfusion throughout the three-dimensional construct. Welding stacked electrospun layers together significantly reduces or minimizes the abrasiveness of the construct boundaries, thereby significantly reducing the risk of an inflammatory response. The three-dimensional construct of the invention is useful in replacing damaged tissue and promoting tissue regeneration as well as in other wide variety of applications.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

### Antibody Immobilization Using CSS

The ability to capture circulating tumor cells (CTCs) provides valuable insight to personalizing cancer treatments. These tumor cells can be captured by functionalizing surfaces with antibodies specific to CTCs. An ideal cell-capturing platform should, in theory, encourage the anchoring of a monolayer of well-oriented antibodies while minimizing the probability of direct cell-scaffold adhesion. Conventional methods rely heavily on hydrophobic interactions for surface functionalization, leading to protein denaturation and loss of function. This example illustrates that engineered cholestryl succinyl silane (CSS) nanostructures can be used to immobilize antibodies in a manner that significantly improves antibody orientation and function. Without being bound by any theory, it is also believed that these cholesterol-based nanostructures significantly reduce or even eliminate direct cell-scaffold interaction, thereby minimizing the risk of non-specific cell capture.

These cholesterol-based nanostructures incorporate anchoring microdomains that appear to substantially mimic naturally occurring membrane microdomains, thereby immobilizing proteins and promoting proper antibody orientation. The performance of CSS nanostructures was compared to electrospun polycaprolactone (PCL) and plasma-treated PCL fibers. While the cholesterol-based nanostructures appeared to anchor less total proteins, in some cases functionalized CSS nanostructures captured nearly six times more cells than functionalized PCL or plasma-treated PCL fibers. The microdomains present within CSS nanostructures demonstrate a surprising and unexpected ability to orient antibodies and maintain protein functionality. As such, cholesterol-based nanostructures are promising platform materials for a wide variety of applications including the capture of CTCs.

Circulating tumor cells (CTCs) have strong diagnostic value as they can be isolated from patients before a primary tumor is ever detected. Furthermore, CTCs are often found when a carcinoma recurs and often persist after the removal of a primary tumor. As such, CTCs have been the focus of intense cell-capture efforts. These tumor cells can be captured from the peripheral blood of patients by engaging ligand-receptor interactions with receptors unique to CTCs. One difficulty in capturing CTCs is that they are present in extremely small numbers; as few as one CTC per 10⁶ or 10⁷ leukocytes. Thus, it is desirable that a cell-capturing platform encourages ligand-receptor interactions whiles minimizing the occurrence of direct cell-scaffold adhesion.

It is well understood that surface functionalization and cell capture can be enhanced or even optimized if antibodies are properly oriented and maintain their native conformational structure, thereby leading to increased bioactivity. Improved antibody performance is desirable in many clinical and laboratory settings, as antibodies are often used as the detection element in increasingly sensitive biosensors, drug delivery systems, and cell-capturing platforms. An ideal cell-capturing platform immobilizes a monolayer of antibodies and minimizes direct cellular adhesion, thereby reducing the probability of capturing non-targeted cells.

This example illustrates immobilization of proteins, such as antibodies, that result in a significantly increased activity. Again without being bound by any theory, it is believed that in some instances, functionalizing surfaces can be based on microdomain interactions between the antibody and the CSS, similar to natural systems. Cholesterol-based materials allow one to engineer nanostructures that mimic naturally-occurring cholesterol microdomains. Such nanostructures can be used to immobilize specific antibodies and capture cells of interest.

In one particular embodiment, cholesteryl succinyl silane (CSS) nanostructures is used to immobilize proteins. As shown herein, CSS immobilizes proteins more efficiently than a simple hydrophobic interaction. Proteins immobilized within the CSS are believed to be better oriented and have shown to maintain better biofunctionality. Without being bound by any theory, CSS is believed to provide a microdomain that immobilizes proteins in a relatively ordered manner compared to immobilization via a simple hydrophobic interaction.

The functionalized surface was created by electrospinning a polymer fiber and attaching the CSS onto the electrospun fibers. In some embodiments, the nanostructure CSS is formed by directly electrospinning a CSS solution, typically in an acidic solution. Thus, in some embodiments, the nanostructure CSS is produced in the absence of a polymer fiber. Generally, the acidic solution that is used to produce the nanostructure CSS (i.e., without a polymer “backbone”) has pH 6 or less, typically pH 5 or less, and often pH 4 or less.

Electrospinning results in a greater area for protein immobilization as the fibers increase the surface to volume ratio. Furthermore, electrospinning is a cheap and facile means by which to produce non-woven scaffolds comprised of fibers with nano and microscale diameters. Without being bound by any theory, it is believed that electrospun polymer fibers with attached CSS interact with immobilized proteins via liquid-ordered microdomains. The performance of these cholesterol-based nanostructures was compared to that of traditional hydrophobic interactions that are conventionally used for protein immobilization. In one particular example, polycaprolactone (PCL) was chosen as a base-line material by which to compare protein immobilization via hydrophobic interactions. PCL is a popular material choice for biomedical applications on its own regard due to its high biocompatibility and acceptable degradation kinetics, not leaving behind an acidic microenvironment. However, PCL's hydrophobicity limits the material's tissue engineering applications, demonstrating low cell loading and leading to decreased cell adhesion and proliferation. PCL can be modified to increase the hydrophilicity and cell-binding ability. However, such a modification is believed to decrease protein immobilization. Thus, plasma-treated PCL fibers were used in this study as a hydrophilic comparison in protein immobilization efficiencies.

Electrospinning cholesterol-based materials can be achieved by providing a solution having a concentration above the entanglement concentration, i.e., the critical micelle concentration or “CMC”. The CMC value is typically unique to the particular nature of CSS. Thus, in one particular embodiment, a CSS solution is prepared in a 69% w/w concentration. At this concentration, it is believed that the elasticity of the solution suppresses the Rayleigh instability and allows for the formation of continuous CSS nanofibers or nanostructure CSS. Natural cholesterol can undergo simple alterations to engineer CSS molecules, that can undergo hydrolysis and partial polymerization. These CSS polymers can, in turn, be electrospun.

In one particular comparative experiment, the present inventors have created electrospun scaffolds from polycaprolactone (PCL), plasma-treated PCL, and cholesteryl succinyl silane (CSS) to compare their abilities to immobilize antibodies via traditional hydrophobic interactions seen for PCL and micro-domain interactions for CSS. The ability to harness micro-domain interaction as a primary method for protein immobilization leads to increased immobilization efficiencies and stronger protein orientation.

Fiber Fabrication:

Three different fiber types were prepared: PCL, plasma-treated PCL, and CSS. A 10% (w/v) solution of PCL was prepared in HFIP (hexafluoro isopropanol). The solution was incubated at room temperature for a minimum of six hours. The sample was briefly vortexed prior to electrospinning. The material was then electrospun with a flow rate of 20 μL/min, a voltage gradient of 12 kV, and a spinneret to ground distance of 12 cm. The fibers were collected on silicon chips that were placed on top of the aluminum foil collector plate. A number of the PCL fiber samples then underwent air-plasma treatment for 10 minutes.

A 69% (w/w) solution of CSS was prepared in a solution of 1 mL of THF with 10 μL of 37% aqueous HCl. The solution was incubated overnight in a 40° C. water bath to create CSS micelles. The solution was then electrospun with a flow rate of 0.5 μL/min flow rate, a voltage gradient of 9 kV, and a spinneret-to-ground distance of 12 cm. The fibers were also collected on Si chips.

Protein Immobilization:

Bovine serum albumin (BSA) was chosen as a model for protein immobilization. BSA is often used to block hydrophobic regions of material surfaces due to its strong affinity of passive immobilization. As such, materials that are hydrophobic should be able to immobilize BSA with relative ease. However, if a material is too strongly hydrophobic it is possible that BSA aggregation occurs as the protein interacts with other BSA molecules. BSA conjugated with Alexa Fluor 488 was purchased from Invitrogen (Carlsbad, Calif.). A 0.001% (w/v) solution was prepared and briefly centrifuged to remove any protein aggregates, which can lead to nonspecific background fluorescence. Electrospun fiber samples on 1 cm² silicon chips were placed in a 12-well plate. Each scaffold was incubated with 20 ug of bovine serum albumin for 90 minutes. The scaffolds were then washed with 1×PBS three times. Fresh 1×PBS was added to each well before the relative intensity was read with the Synergy 2 SL Luminescence Microplate Reader (BioTek, VT). Samples were stored in the dark at room temperature between reads. The ability of each scaffold to sequester the proteins was evaluated for six days.

Anti-CD20 Immobilization and Granta-22 B-Cell Lymphoma Cell Capture:

Each 0.25 cm² scaffold was placed in a single well of a 48-well plate. The scaffolds were then prewashed with PBS (3×). Each scaffold was then incubated in a dilute solution of anti-CD20 (10 μg/mL) for 90 minutes. The scaffolds were then washed with PBS prior to a 60-minute incubation in a 0.1% BSA in 1×PBS solution. The scaffolds were then washed again prior to seeding Granta-22 B-cell lymphomas at a concentration of 2×10⁵ cells per scaffold. The scaffolds then incubated for 45 minutes to allow for cell capture. After cell capture the scaffolds were washed again and then subjected to a 15-minute incubation in 4% paraformaldehyde. The captured cells were then treated with Triton-x prior to being stained with Alexa Fluor phalloidin to image cell actin and ProLong® Gold Antifade with DAPI to image the cell nuclei (Life Technologies, USA). The capture cells were imaged the following day with fluorescent microscopy (Nikon).

Statistical Analysis:

Student's t-test was computed between each fiber type to determine statistical significance for cell capture efficiency.

Results and Discussion:

Incorporating nanostructured surfaces for the capture of circulating tumor cells can be helpful in minimizing the probability of capturing non-targeted cells. A PCL surface can be made less conducive to cell attachment by electrospinning the polymer. Electrospun plasma-treated PCL demonstrated high wettability, increasing the probability of cell attachment that occurs independently of an interaction between the anchored antibody and the targeted cell. Electrospun CSS had a wettability contact angle of 136°. Similar non-wettable, nanostructured surfaces have shown a significantly improved protein immobilization while substantially reducing direct cellular adhesion.

In order to evaluate the ability of the electrospun fibers to immobilize proteins of interest, bovine serum albumin (BSA) conjugated to Alexa Fluor 488 was immobilized onto the three scaffold types. Relative intensities (RI) of the fluorescently conjugated BSA were collected over a six-day period. See FIG. 2. The RI of the conjugated BSA increased over time on the electrospun PCL fibers. This may be explained by the aggregation of BSA on the electrospun PCL, resulting in extremely high RIs. High levels of aggregated protein were present (over the first 90 minutes of protein immobilization on PCL fibers, protein aggregates were visible to the naked eye). However, the protein aggregate would not encourage protein-cell interaction, as many of the protein active sites would be blocked by the protein aggregate. The increased RI may be an artifact of non-specific background florescence seen in protein aggregation.

The relative intensities for plasma-treated PCL or the CSS scaffolds declined the day following immobilization but then stabilized for the remainder of the 6-day run. As such, the relative intensities were converted to protein concentrations. It was found that CSS and plasma-treated PCL were able to immobilize similar levels of protein over the six days (approximately 0.6 ug per scaffold). The assumption that protein immobilization is driven simply by hydrophobic interaction would predict that the CSS fibers would have immobilized the greatest number of protein due to its extremely hydrophobic nature. However, this was not found to hold true. It is believed that the sterol rings within the CSS molecule help anchor proteins to the fibers, allowing for the immobilization of a single monolayer of protein.

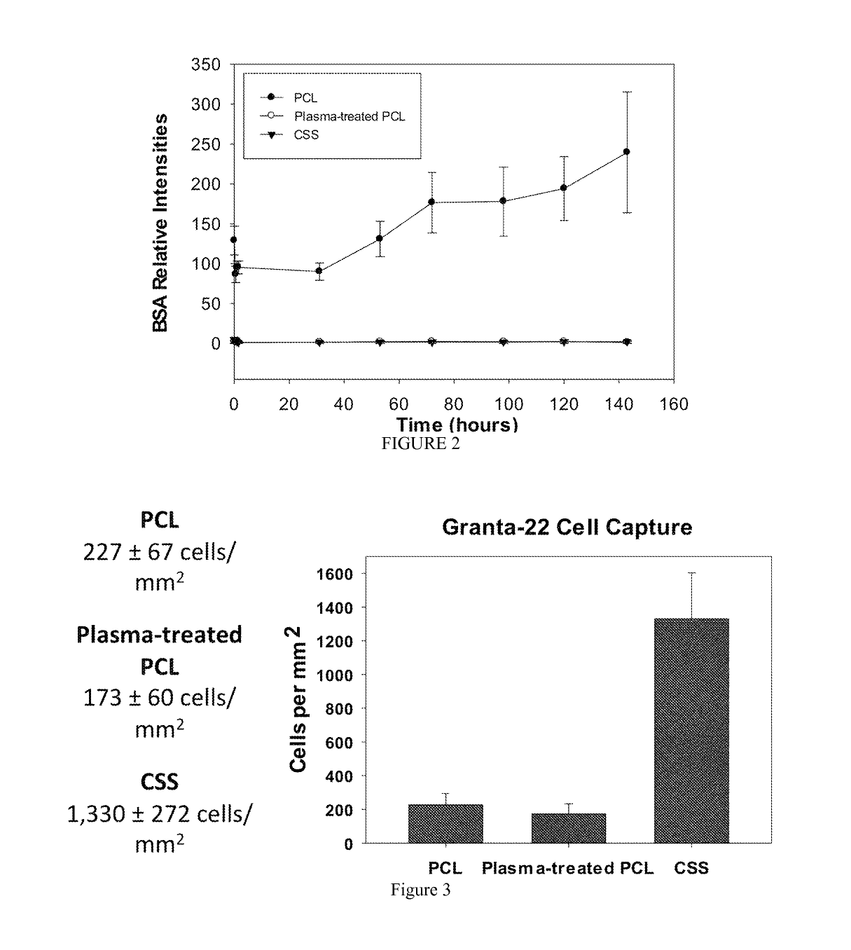

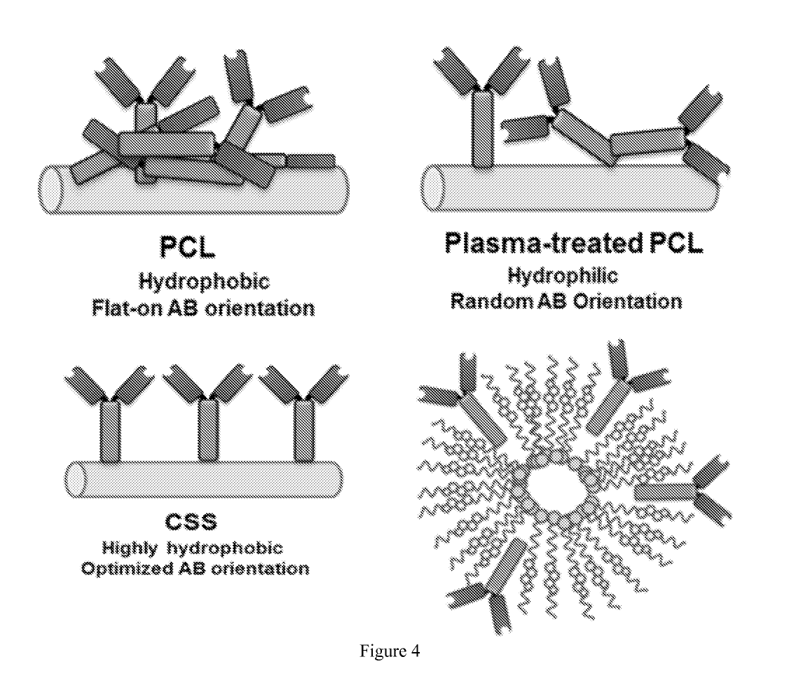

After quantifying protein immobilization, the fibers were investigated for their abilities to capture cells. It was found that CSS was able to capture substantially more cells than either the PCL or plasma-treated PCL fiber (FIGS. 3 and 4, where FIG. 4 is cell stains confirming the data presented in FIG. 3). PCL fibers and plasma-treated PCL captured 227±67 cells/mm² and 173±60 cells/mm², respectively. CSS fibers captured 1,330±272 cells/mm², nearly a six-fold increase in capture efficiency (p<0.005). Histological slides (not shown) also indicated that the cells on CSS fibers showed increased actin filament formation as compared to cells seen on either the PCL or plasma-treated PCL fibers. Increased presence of actin filaments on the CSS scaffolds seems indicative of stronger interactions between the anchored antibodies and the targeted cells.

Even though plasma-treated PCL fibers and CSS fibers immobilized a similar concentration of protein, CSS fibers were capable at least a six-fold increase in cell capture. Thus, these cholesterol based fibers appear to be promoting proper antibody orientation. The inherent properties of the three materials can explain the protein immobilization pattern that is seen with each material (FIG. 4). For example, the strong hydrophobicity of PCL promotes protein aggregation. Thus, these scaffolds can immobilize high numbers of protein but are ultimately inefficient at capturing cells of interest. On the other hand, plasma-treated PCL's hydrophillicity immobilizes a lower amount of protein but is substantially incapable of promoting proper orientation. CSS nanostructures on the other hand are believed to provide anchoring microdomains that interact with the hydrophobic portions of the antibody's Fc region. This would lead to the immobilization of a single, uniform layer of antibodies that are properly oriented. Such a layer of antibodies would then be capable of highly efficient cell capture, as was shown within this study.

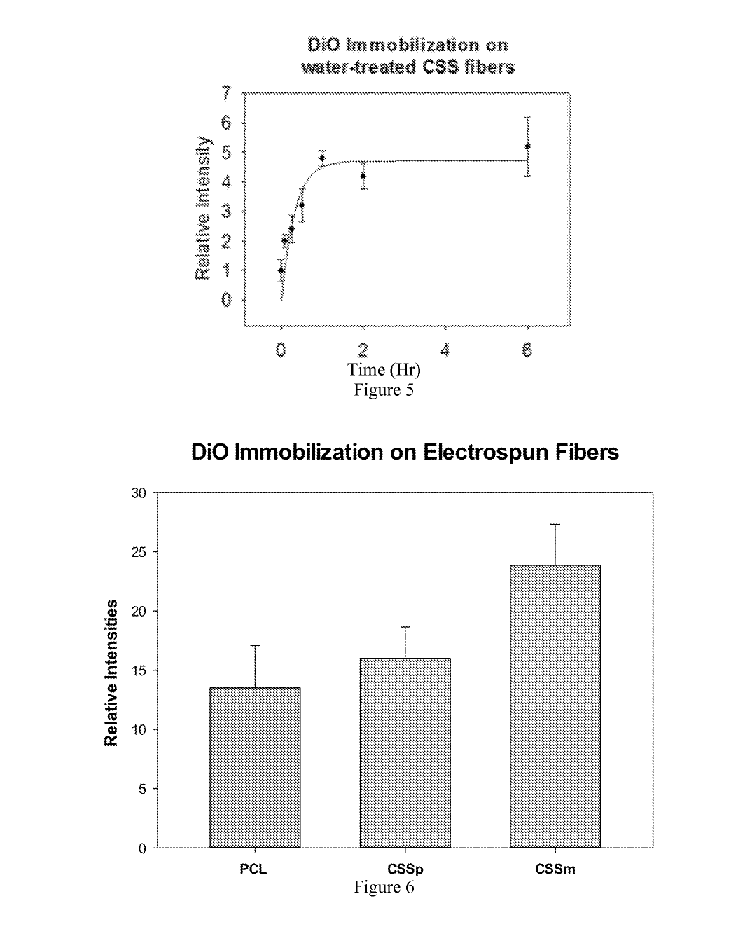

Without being bound by any theory, it is believed that after the CSS fibers are exposed to water, the polymerizable head is found on the outside of the fiber while the hydrophobic sterol tail is tucked within the fiber. Figure labeled “DiO Immobilization on water-treated CSS fibers” (FIG. 5) helps illustrate this point: that when the fibers are exposed to water they undergo a rearrangement that then enhances the fiber organization for protein/antibody immobilization.

CSS nanostructures are well suited for cell capture for at least two reasons. First, the microdomains provide an anchoring site for protein immobilization. This helps assure that a well oriented monolayer of protein is immobilized on the substrate surface. Second, the nanostructures increase CSS's hydrophobicity, minimizing the risk of non-specific cell adhesion. This is particularly important when a small subset of cells is of interest (as in the case with circulating tumor cells). Thus, CSS nanostructures can be used in a variety of application including in medical devices to effectively capture and isolate CTCs.

### Example 2

### PCL: CSS Fibers

In this example, PCL fibers were formed by electrospin process using a 10% w/v PCL solution. In some instances, the resulting PCL fibers were treated with air-plasma for 10 minutes. And the air-plasma treated fibers were contacted (or incubated) with (i) polymerized CSS, in which CSS was polymerized on its own overnight, and the resulting solution was then incubated with plasma-treated PCL fibers overnight, or (ii) CSS monomers, in which a solution of CSS (0.1%, 1% or 5%) monomer was allowed to form nanostructure CSS on PCL fiber overnight.

Experiment similar to that in Example 1 was performed with DiO to determine simulated protein immobilization. The results are shown in FIG. 6, where PCL is air-plasma treated PCL, CSSp is air-treated PCL incubated with CSS polymer, and CSSm is air-treated PCL incubated with CSS monomer.

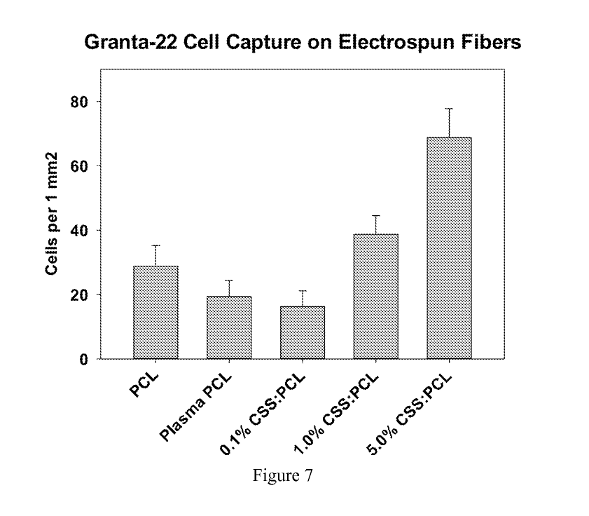

Cell capture experiments were conducted by immobilizing antibody (αCD20) to capture Granta-22 B-cell lymphomas in PCL polymer, PCL polymer treated with air-plasma, air-plasma treated PCL incubated with 0.1%, 1% and 5% CSS monomer solution. The results are shown on FIG. 7.

Table 1 below shows different cell surface markers for different metastatic breast cancer cell lines. Using the compositions and methods disclosed herein, cell surface markers can be targeted using appropriate antibody to capture different metastatic breast cancer cells.

TABLE 1
Cell surface markers in different breast cancer cells (+ =
high expression; − = low expression):

| | | Metastatic Breast Cancer Cell Lines | | |
|---|---|---|---|---|
| | | MDA-MB-231 | SK-BR-3 | MCF-7 |
| Cell Surface Markers | EpCAM | −− | + | + |
| | CK19 | − | ++ | + |
| | Vimentin | ++ | − | − |
| | CD44 | + | −− | −− |

FIG. 8 shows results of about 1,000 MDA-MB-231 cells that were exposed to CSS chips with immobilized antibodies. The left panel shows absolute number of cells captured with 5 different combinations of capture agents, and the right panel shows cells captured by antibodies that were cell specific. The antibodies were specific for cell surface markers α-Vimentin, E-Selectin, and the EpCam/α-Vimentin combination.

### Example 3

### Three-Dimensional In Vitro Tissue Construct for Complex Cell Studies

In vitro cell studies are typically performed on a 2D platform (such as tissue culture plates). These platforms give scientists a poor idea of how cells interact within their native 3D environments. Currently matrigels are commercially available that allow for 3D cell seeding. However, these matrigels often lack the micro- and nano-structures that are present within natural tissue samples. The lack of a well-engineered 3D construct for in vitro cell studies often make animal studies necessary, significantly increasing study costs while not ensuring accurate insight for disease states that occur in vivo. To overcome these shortcomings, the present inventors have have engineered a three dimensional construct that: 1) is easy to fabricate, 2) is cheap to produce, and 3) can provide a platform for multiple cell types to interact in a manner that is more indicative to in vivo tissue environments.

The three-dimensional in vitro tissue construct is fabricated by electrospinning polymers of interest that act as the backbone for the construct. Numerous polymers can be chosen based on their mechanical properties, biocompatibility, and degradation rates. Microchannels are then added to the scaffolds to help promote cellular migration and the transportation of nutrient/waste through the scaffold. Multiple layers of electrospun scaffold are then welded together, creating a singular construct with multiple cell seeding layers/microenvironments. This construct design allows for quick and cheap fabrication of 3D electrospun constructs.

A three-dimensional construct was engineered as a novel in vitro environment for complex cell studies. The construct has a number of variables that can be customized depending on the type of tissue environment that is being replicated within the construct. For example, the electrospun fibers can be randomly oriented or aligned; dependent on what type of tissue environment is being synthetically replicated. The 3D constructs were fabricated by electrospinning 2D scaffolds with either aligned or randomly oriented fibers. The resulting scaffolds were then cut into 2D discs. The individual scaffolds were then perforated with microchannels (to allow for nutrient perfusion through the 3D construct). Multiple electrospun layers were then stacked one on top the other to create a 3D construct. The scaffold layers were then welded together at discrete points along the outer perimeter of the construct. Cells have been seeded on the top layer of the construct (cell proliferation throughout the construct is encouraged by the inclusion of the microchannels). Such a three-dimensional scaffold allows for a more accurate representation of in vivo tissue conditions. This tool can cheaply and quickly allow researchers to study interactions (cell-cell, cell-drug, etc.) that occur within a 3D environment that more closely resembles in vivo tissue environments.

The inappropriate design of commercially available in vitro tissue culture systems have made it difficult to interpret results in a way that can lead to understanding of interactions that occur in vivo. Currently it is possible to study human cells within simple in vitro systems but the results often lead to expensive animal studies that give scientists a better idea of what is happening within the organism. These animal studies are also inherently limiting in that they do not provide insight within human tissue systems. As such, a three-dimensional in vitro construct that allows for the study of human cells lines would provide an invaluable research tool for the understanding of interactions that occur within a biomimetic environment. Such a construct can be used for understanding the cross-talk between different cell types, gaining insight of cancer development and progression, studying drug-tissue interactions, etc.

A number of difficulties arise when building 3D in vitro constructs: 1) perfusion of nutrients and waste through the construct, 2) channels that allow for cellular proliferation through the construct and 3) delamination of individual electrospun scaffolds. The 3D polymer of the invention overcomes these difficulties. Thus, the construct of the invention can be used in an in vitro system to study cell interactions in a more natural-like environment.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A composition comprising:
a nanostructure cholesteryl succinyl silane (CSS); and
an antibody immobilized on said nanostructure CSS.

2. The composition of claim 1, wherein said antibody is adapted for selectively capturing a cell.

3. The composition of claim 2, wherein said cell is a circulating tumor cell.

4. The composition of claim 3, wherein said circulating tumor cell is breast cancer cell, leukemic cell, lymphoma cancer cell, metastatic breast cancer cell, or any other metastatic cancer cell.

5. The composition of claim 1 further comprising a non-woven polymer fiber, and wherein said nanostructure CSS is attached to said non-woven polymer fiber.

6. The composition of claim 5, wherein the diameter of said non-woven polymer fiber is substantially similar to extracellular matrix.

7. The composition of claim 5, wherein said non-woven polymer fiber is made from a polymer comprising poly(caprolactone), polyvinyl alcohol, polylactic acid, a copolymer comprising the same, or any combination thereof.

8. The composition of claim 1, wherein a monomeric unit of said cholesteryl succinyl silane is of the formula: A-B-C, wherein A is cholesterol, B is succinyl unit, and C is a silane unit.

9. The composition of claim 8, wherein said silane unit is a moiety of the formula:
—R^{a}—Si(OR^{b})₃, wherein R^{a} is alkylene, and each of R^{b} is independently hydrogen or alkyl.

10. A method for producing a composition comprising a nanostructure cholesteryl succinyl silane (CSS) and an antibody immobilized on said nanostructure CSS, said method comprising:
providing an acidic solution of cholesteryl succinyl silane having a critical micelle concentration;
producing a nanostructure CSS from said acidic solution of cholesteryl succinyl silane using an electrospinning process; and
immobilizing an antibody to said nanostructure cholesteryl succinyl silane to produce said composition.

11. A method for detecting the presence of a cancer in a subject, said method comprising:
contacting a fluid sample of the subject with a composition comprising:
a nanostructure cholesteryl succinyl silane (CSS); and
a cancer cell antibody immobilized on said CSS; and
determining the formation of a complex between said cancer cell antibody and a cancer cell, wherein the presence of said complex is an indication that said subject has a cancer.

12. The method of claim 11, wherein said cancer cell comprises a circulating cancer cell.

13. The method of claim 11, wherein said composition further comprises a non-woven polymer fiber, and wherein said nanostructure CSS is attached to said non-woven polymer fiber.
